(19) United States
(12) Patent Application Publication
(10) Pub. No.: US 2016/0346210 A1
(43) Pub. Date: Dec. 1, 2016

(54) MEDICAL MAGNETIC PELLETS

(71) Applicant: bio-Peptide Enhancer Tech Inc., Ltd., New Taipei City (TW)

(72) Inventor: Chao-Hsiang CHANG, New Taipei City (TW)

(21) Appl. No.: 15/166,285

(22) Filed: May 27, 2016

(30) Foreign Application Priority Data
May 29, 2015 (TW) 104117472

(51) Int. Cl. A61K 9/16; A61K 47/22; A61K 47/12

(52) U.S. Cl. CPC A61K 9/1611; A61K 9/1652; A61K 9/1617; A61K 47/12; A61K 47/22

(57) ABSTRACT

A medical magnetic pellet is provided. The magnetic pellet includes a porous matrix and magnetic powder distributed in the porous matrix. Moreover, an active substance also can be added to be distributed in the matrix. The porous matrix of the magnetic pellet can carry active substances or absorb target substances.

### CROSS-REFERENCE TO RELATED APPLICATION

This application claims the priority benefit of Taiwan application serial no. 104117472, filed May 29, 2015, the full disclosure of which is incorporated herein by reference.

### BACKGROUND

### Field of Invention

The disclosure relates to a drug carrier. More particularly, the disclosure relates to a magnetic drug carrier.

### Description of Related Art

The available magnetic carriers are only applied in laboratory testing reagents on a small scale. The related products are often called as magnetic beads. The magnetic beads are usually made by chemically modifying a single magnetic particle. For example, individual magnetic particle can be respectively modified by an antibody and then conjugated with a drug molecule to form the so-called immune magnetic beads, or can be directly coated by a drug. The magnetic beads above usually have a diameter of 20-50 μm. In some other examples, a polymer can be used to coat the magnetic powder. Or, the magnetic powder can be dispersed in a polymer solution having a low concentration first, and the solution is sprayed on cores to form magnetic beads having a diameter of 30-100 μm.

Since the prices of the various magnetic beads above are too expensive, and thus are rarely used in clinical applications, such as intramuscular, subcutaneous or intravenous injection, or oral dosage forms. Therefore, a novel magnetic carrier is needed to solve the problems above.

### SUMMARY

In one aspect, a medical magnetic pellet is provided. The magnetic pellet comprises a first matrix and magnetic powder. The first matrix has pores and contains a modified cellulose, a starch or a combination thereof. The magnetic powder is distributed in the first matrix and made from a material which is magnetic or can be induced to be magnetic.

According to one embodiment, the content of the first matrix may be 2.5-50 parts by weight.

According to another embodiment, the content of the magnetic powder may be 5-25 parts by weight.

According to yet another embodiment, the pores have a diameter of 0.1-20 μm.

According to yet another embodiment, the modified cellulose may be hydroxypropyl methyl cellulose (HPMC), hydroxypropyl cellulose (HPC), hydroxyethyl cellulose (HEC), microcrystalline cellulose (MCC), or any combinations thereof.

According to yet another embodiment, a material of the magnetic powder is an element, an oxide, an alloy, a salt, or any combinations thereof that contains at least one of Fe, Co, Ni, Nd and Sr.

According to yet another embodiment, the magnetic pellet further comprises a first active substance distributed in the first matrix, the pores or a combination thereof, wherein the first active substance has at least a biological activity.

According to yet another embodiment, a content of the first active substance is 30-70 parts by weight.

According to yet another embodiment, the magnetic pellet further comprises a shell layer coating on the first matrix.

According to yet another embodiment, a material of the shell layer is a monosaccharide, a disaccharide, a sugar alcohol, an enteric polymer, or a fatty acid or an ester thereof having 5-50 carbons.

In another aspect, a medical magnetic composite pellet is provided. The magnetic composite pellet comprises a second matrix and any one of the magnetic pellets above to be distributed in the second matrix.

According to one embodiment, the second matrix is made from water soluble small molecules, comprising a monosaccharide, an amino acid, an organic acid, a sugar alcohol, or a combination thereof.

According to yet one embodiment, the magnetic composite pellet further comprises a second active substance distributed in the second matrix, wherein the second substance having at least a biological activity.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a cross-sectional diagram showing a magnetic pellet according to an embodiment of this invention;

FIG. 2 is a cross-sectional diagram showing a magnetic pellet having a shell layer according to an embodiment of this invention; and

FIG. 3 is a cross-sectional diagram showing a magnetic composite pellet according to an embodiment of this invention.

### DETAILED DESCRIPTION

Accordingly, a medical magnetic pellet and a preparation method thereof are provided. In the exemplary description below, the exemplary structure and preparation method of the magnetic pellet above will be introduced. To more detailed and comprehensively describe this invention, the description below illustrates the embodiments of this invention, but this is not the only way to practice or use the embodiments of this invention. The disclosure comprises many features and steps of how to construct and operate these embodiments. However, other embodiments also may be used to acquire the same or equivalent functions and steps.

### Magnetic Pellets

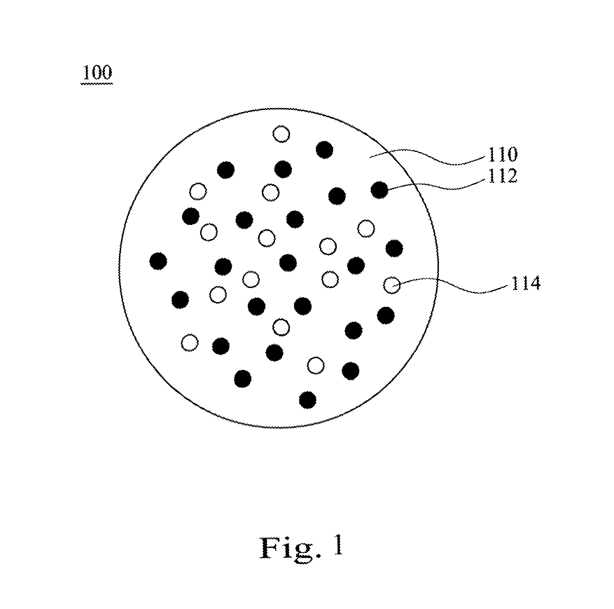

Please refer to FIG. 1. FIG. 1 is a cross-sectional diagram showing a magnetic pellet according to an embodiment of this invention. In FIG. 1, the magnetic pellet 100, used as a carrier, comprises a first matrix 110, and magnetic powder 112 distributed in the first matrix 110.

The first matrix 110 has a porous structure. The content of the first matrix 110 is about 2.5-50 parts by weight, such as 2.5, 3, 5, 7, 10, 15, 20, 25, 30, 35, 40, 45 or 50 parts by weight. The material of the first matrix 110 may contains a modified cellulose, a starch, or a combination thereof. The modified cellulose may be hydroxypropyl methyl cellulose (HPMC), hydroxypropyl cellulose (HPC), hydroxyethyl cellulose (HEC), microcrystalline cellulose (MCC), or any combinations thereof. For example, 2-10 wt % of HPC aqueous solution having higher viscosity may be used as an adhesive, and then mixed with other water soluble modified celluloses to be the excipient.

The content of the magnetic powder 112 is about 5-25 parts by weight, such as 5, 7, 10, 15, 20, or 25 parts by weight. The material of the magnetic powder 112 are magnetic or can be induced to be magnetic. Therefore, the magnetic powder may made from a ferromagnetic material or a ferrimagnetic material, such as an element, an oxide, an alloy, a salt, or any combinations thereof that contains at least one of Fe, Co, Ni, Nd and Sr.

According to an embodiment, the magnetic pellet 100 may further comprise a first active substance 114 distributed in the first matrix 110. The content of the first active substance 114 is about 30-70 parts by weight, such as 30, 35, 40, 45, 50, 55, 60, 65, or 70 parts by weight. The material of the first active substance 114 may include any available synthesized compounds or extracts from natural products that have biological activity, such as nutritional supplements or drugs, or biological materials, such as enzymes, organelles, or cells.

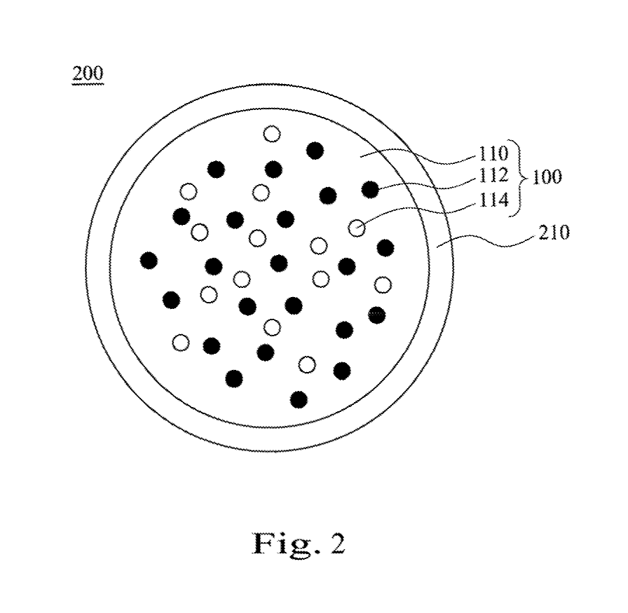

Please refer to FIG. 2. FIG. 2 is a cross-sectional diagram showing a magnetic pellet having a shell layer according to an embodiment of this invention. In FIG. 2, the magnetic pellet 200 include the magnetic pellet 100 in FIG. 1 and a shell layer 210 coating on the magnetic pellet 100. The structure of the magnetic pellet 100 has been described above, and hence the related description is omitted here.

In pharmaceuticals, the shell layer 210 may be a sugar coating or a film coating to provide new functions to the magnetic pellet. For examples, the shell layer 210 can further provide functions of isolation, controlled release, or controlled adsorption. Except improving taste, the sugar coating may increase the stability and hardness of the magnetic pellet 100. The film coating can be divided into time release type, pH dependent type, moisture resistant type, elastic and compression-resistant type, long-acting type, and large intestine release type. Therefore, in addition to increase the stability of the magnetic pellet 100, the shell layer 210 can be controlled to be discomposed in a selected environment, so that the magnetic pellet 100 can contact the outside environment to release active substances or adsorb substances.

When the shell layer 210 is a sugar coating, the material may be a monosaccharide, a disaccharide, a sugar alcohol, an enteric polymer, or a fatty acid or an ester thereof having 5-50 carbons. In addition, the material of the shell layer 210 may further comprise any one of the first active substance 114 above.

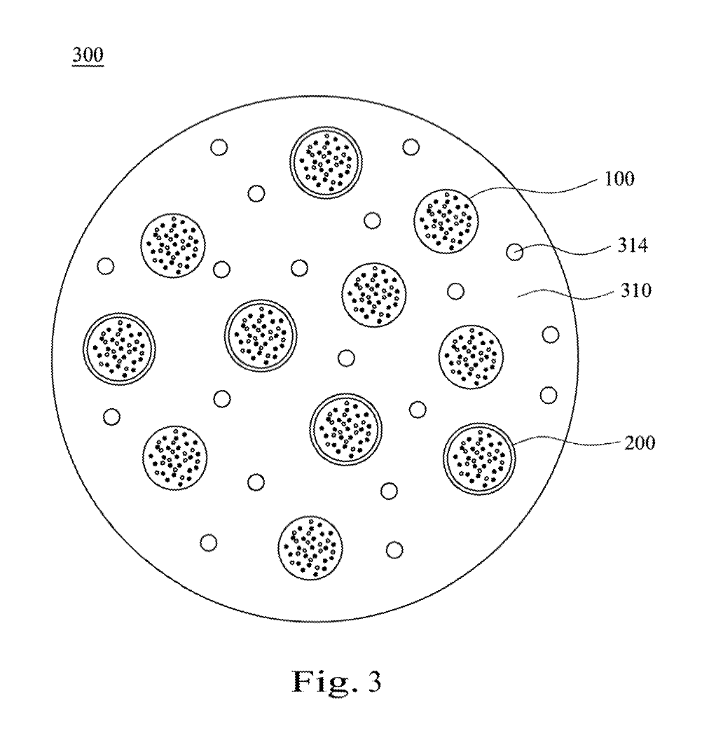

Please refer to FIG. 3. FIG. 3 is a cross-sectional diagram showing a magnetic composite pellet according to an embodiment of this invention. In FIG. 3, the magnetic composite pellet 300 comprises many magnetic pellet 100 having no shell in FIG. 1 and magnetic pellet 200 having the shell layer 210 in FIG. 2 distributed in the second matrix 310, but is not limited thereto. That is, magnetic pellet 100 and magnetic pellet 200 also may be used alone to form the magnetic composite pellet 300.

The magnetic pellet 100, magnetic pellet 200, or a combination thereof are distributed in the second matrix 310 to form the magnetic composite pellet 300. This not only increase the stability of the magnetic pellet 100 and magnetic pellet 200, but also can stepwise release the active substances in the magnetic pellet 100 and/or magnetic pellet 200. Therefore, the material of the second substrate 310 may be water soluble small molecules, such as monosaccharides, amino acids, organic acids, or sugar alcohols.

Moreover, a second active substance 314 may be chosen to be distributed in the second matrix 310. Similar to the first active substance 114, the material of the second active substance 314 may include any available synthesized compounds or extracts from natural products that have biological activity, such as nutritional supplements or drugs, or biological materials, such as enzymes, organelles, or cells.

According to an embodiment, the diameters of the various magnetic pellets above are about 20-3,000 μm. For example, when the diameter of the magnetic pellet is 20-70 μm, the magnetic pellet can enter the circulatory system, interstitial tissue, or organ system via intravenous injection or other injection ways. Therefore, the magnetic pellets can stay in the circulatory system to achieve the purposes of treatment or inspection.

When the diameter of the magnetic pellets is 150-3,000 μm, the magnetic pellets can enter the digestive tract, i.e. from throat, then pass through esophagus, stomach, duodenum, jejunum, ileum and colon, as well as finally discharge from the anus. Accordingly, it can be known that the magnetic pellets can stay in any organs of the digestive tract to achieve the purposes of treatment or inspection. When the magnetic pellets are discharged from a human body, the magnetic pellets can be easily separated from other excreta by using a magnetic device as the magnetic pellets can roughly maintain their structure.

### Preparation Method of Magnetic Pellets

When the material of the first matrix contains a modified cellulose, 5 wt % of a modified cellulose aqueous solution, such as hydroxypropylmethyl cellulose (HPMC) aqueous solution, may be added into a mixture of microcrystalline cellulose (MCC), magnetic powder, and an active substance, and then uniformly mixed to obtain a magnetic pellet's mixture.

Next, extrusion and spheronization are performed to obtain magnetic pellets. The hole diameter of an extrusion board used in extrusion and the rotation rate in the spheronization step can be adjusted to obtain magnetic pellets with various diameters.

After extrusion and spheronization, the subsequent drying method is determined according to the addition amount of the modifier cellulose. When the content of the modified cellulose is higher, fluid-bed drying is performed to remove the excess water to obtain solid magnetic pellets. When the content of the modifier cellulose is lower, shade drying is performed to remove water to obtain porous magnetic pellet.

If a water soluble substance is added in the magnetic pellet's mixture, the shade-dried magnetic pellets may be further immersed in water for a period of time. After the water soluble substance is dissolved in the water and then dried by heat, magnetic pellets with higher porosity may be obtained. The pore diameters of the obtained porous magnetic pellets may as large as 0.1-20 μm.

The structure strength of the magnetic pellets is tested to measure the greatest pressure that the magnetic pellet can stand without being crushed. The bearable pressure of the solid magnetic pellets is about 100-400 mPa. The bearable pressure of the porous magnetic pellets is about 20-100 mPa.

When the material of the first matrix comprises water-insoluble long-chain fatty acids or esters thereof, the preparation method is described below. The long-chain fatty acids or esters thereof are heated to be melted. Magnetic powder and active substances are added into the melted fatty acids or esters thereof and then uniformly mixed. After cooling down, the solid is smashed and sieved to obtain magnetic pellets with desirable sizes.

### Magnetism Measurement of Magnetic Pellets

In the embodiments below, the magnetism measurement of magnetic pellets is described below. 6 grains of magnetic pellets before or after being magnetized were taken first. Magnetism of the 6 magnetic pellets were measured by a handheld probe type gauss meter and then averaged to obtain an average value thereof. The magnetic force is an action at a distance, and the magnetic dipole is a vector. Therefore, the measured magnetism would be different as the measuring direction and distance are different. Accordingly, only the maximum measured magnetism were taken. The unit of the magnetism is gauss (G).

### Embodiment 1

### Solid Magnetic Pellets

In this embodiment, the magnetic pellets 100 having a solid structure in FIG. 1 were prepared by extrusion and spheronization. The material of the magnetic powder was SrO.6Fe₂O₃ (from JASDI Magnet, Taiwan). The material of the first matrix was MCC and HPMC. The preparation method is described below.

5 wt % of HPMC aqueous solution was prepared, and then added to a mixture of SrO.6Fe₂O₃ powder and MCC, and then were all uniformly mixed together. Next, extrusion and spheronization was performed to obtain wet magnetic pellets with various diameters in a range of 0.2-2.0 mm. Then, fluid-bed drying was performed to dry the wet magnetic pellets.

The starting materials and the outward appearance of the dried magnetic pellets are listed in Table 1. From Table 1, it can be known that when the total content of the HPMC and MCC was at least 50 wt %, the extrusion and spheronization can be used to form grains.

TABLE 1
The starting materials and the outward appearance of obtained
magnetic pellets.

| | SrO•6Fe₂O₃ | HPMC | | diameter | Outward |
|---|---|---|---|---|---|
| Example | (g) | (g) | MCC (g) | (mm) | appearance |
| 1-1 | 500 | 50 | 450 | 2.0 | Spherical |
| 1-2 | 500 | 50 | 450 | 1.2 | Spherical |
| 1-3 | 500 | 50 | 450 | 1.0 | Spherical |
| 1-4 | 500 | 50 | 450 | 0.8 | Spherical |
| 1-5 | 500 | 50 | 450 | 0.6 | Spherical |
| 1-6 | 500 | 50 | 450 | 0.4 | Quasi- |
| | | | | | spherical |
| 1-7 | 500 | 50 | 450 | 0.3 | Quasi- |
| | | | | | spherical |
| 1-8 | 500 | 50 | 450 | 0.2 | Quash- |
| | | | | | spherical |

All of the obtained magnetic pellets before being magnetized can be attracted by a magnetic board having a magnetic field of 200 G. Then, the magnetism of the non-magnetized magnetic pellets was measured to obtain the maximum magnetism values. Next, the magnetic pellets are magnetized by a magnetizing machine, and the magnetism thereof was then measured again. Furthermore, the magnetic pellets filled in capsules having a size of zero number (outer diameter 7.65 mm, length 21.7 mm, volume 0.68 mL) until the magnetic pellets could not rock, the entire capsules were magnetized and then measured the magnetism thereof again. The obtained data are listed in Table 2 below.

TABLE 2
Magnetism of magnetic pellets before and after magnetization.

| | Magnetic Pellet | | | Magnetic pellets in | |
|---|---|---|---|---|---|
| | | Magnetism (G) | | capsule zero | |
| | Diameter | non- | | Filling | Magnetism |
| Ex. | (mm) | magnetized | magnetized | amount (mg) | (G) |
| 1-1 | 2.0 | 5.0 | 107 | 493 | 143 |
| 1-2 | 1.2 | 4.7 | 101 | 541 | 159 |
| 1-3 | 1.0 | 4.3 | 100 | 563 | 163 |
| 1-4 | 0.8 | 4.1 | 87 | 575 | 168 |
| 1-5 | 0.6 | 3.9 | 73 | 593 | 179 |
| 1-6 | 0.4 | 3.3 | 51 | 599 | 173 |
| 1-7 | 0.3 | 3.3 | 49 | 607 | 179 |
| 1-8 | 0.2 | 3.1 | 47 | 613 | 175 |

From Table 2, it can be known that the diameter of the magnetic pellets could affect the magnetism, and larger magnetic pellets had greater magnetism. Comparing with the magnetism of magnetic powder (<3 G), the magnetism of the non-magnetized magnetic pellet was at least 3 G, and the magnetism of the magnetized magnetic pellet could be increased to at least 40 G, which is 20 times of the magnetism of the magnetic powder. When the capsule was filled by the magnetic pellets and then magnetized, the magnetism thereof could be further increased to at least 140 G.

### Embodiment 2

### Solid Magnetic Pellets Having a Shell Layer

In this embodiment, the magnetic pellets of examples 1-1 to 1-8 were coated by a shell layer to obtain the magnetic pellets 200 having a shell layer 210 in FIG. 2. The material of the shell layer is HPMC. Magnetization was then performed to obtain the magnetic pellets of examples 2-1 to 2-8. The obtained data are listed in Table 3. Comparing Tables 2 and 3, it can be known that whether the magnetic pellet was coated by a shell layer or not, the magnetism thereof was basically the same. That is, the shell layer does not affect the magnetism of the magnetic pellets basically.

TABLE 3
The magnetism of magnetic pellets having a shell layer. The
content of the shell layer is 10 wt % of the magnetic pellet core.

| Example | Magnetism (G) | Outward appearance |
|---|---|---|
| 2-1 | 105 | Spherical |
| 2-2 | 99 | Spherical |
| 2-3 | 103 | Spherical |
| 2-4 | 92 | Spherical |
| 2-5 | 75 | Spherical |
| 2-6 | 53 | Quasi-spherical |
| 2-7 | 52 | Quasi-spherical |
| 2-8 | 49 | Quasi-spherical |

### Embodiment 3

### Effect of Material of Magnetic Powder on the Magnetism of the Magnetic Pellets

In this embodiment, the materials of the magnetic powder were changed to observe the effect on the magnetism of the magnetic pellets. The materials of the magnetic powder include reduced iron (i.e. elemental iron), NdFeB alloy (Nd₂Fe₁₄B), Fe₄(P₂O₇)₃ (an edible iron agent), Fe₂O₃, and SrO.6Fe₂O₃.

The material of the first matrix is a long-chain saturated fatty acid having 46 carbons. Since the melting temperature of this fatty acid is greater than 60° C. and this fatty acid is insoluble in water, the preparation method of the magnetic pellets was heat melting and then smashing. That is, the fatty acid was heated until it is melted, magnetic powder was then added and mixed with the melted fatty acid. After cooling down, the solid was smashed and sieved to obtain the magnetic pellets with desired size. The results are listed in Table 4.

TABLE 4-1
Data of magnetic powder

| | | | Magnetized |
|---|---|---|---|
| Magnetic powder's material | Example | Diameter (μm) | magnetism (G) |
| Reduced iron* | 3-1 | 75-150 | 0 |
| | 3-2 | | |
| | 3-3 | | |
| NdFeB alloy | 3-4 | 30-50 | 215 |
| | 3-5 | | |
| | 3-6 | | |
| Fe₄(P₂O₇)₃* | 3-7 | 75-150 | 0 |
| | 3-8 | | |
| | 3-9 | | |
| Fe₂O₃ | 3-10 | 75-150 | 0 |
| | 3-11 | | |
| | 3-12 | | |
| SrO•6Fe₂O₃ | 3-13 | 1-5 | 295 |
| | 3-14 | | |
| | 3-15 | | |

*an edible iron agent listed by Ministry of Health and Welfare, Taiwan

TABLE 4-2
Effect of magnetic powder's material on magnetism of
magnetic pellets.

| | | MP*:Sat'd | | | |
|---|---|---|---|---|---|
| Magnetic | | FA | | Non- | Magnetized |
| powder's | | (weight | Dia. | magnetized | magnetism |
| material | Ex. | ratio) | (μm) | magnetism (G) | (G) |
| Reduced iron | 3-1 | 1:1 | 180-250 | 0 | 97 |
| | 3-2 | 2:1 | | | |
| | 3-3 | 4:1 | | | |
| NdFeB alloy | 3-4 | 1:1 | 180-250 | 237 | 575 |
| | 3-5 | 2:1 | | | |
| | 3-6 | 4:1 | | | |
| Fe₄(P₂O₇)₃ | 3-7 | 1:1 | 180-250 | 0 | 127 |
| | 3-8 | 2:1 | | | |
| | 3-9 | 4:1 | | | |
| Fe₂O₃ | 3-10 | 1:1 | 180-250 | 0 | 203 |
| | 3-11 | 2:1 | | | |
| | 3-12 | 4:1 | | | |
| SrO•6Fe₂O₃ | 3-13 | 1:1 | 180-250 | 335 | 892 |
| | 3-14 | 2:1 | | | |
| | 3-15 | 4:1 | | | |

*abbreviation of magnetic powder

In Table 4-2, the magnetic pellets in examples 3-13 to 3-15 using magnetic powder made of SrO.6Fe₂O₃ had the greatest magnetism. Next, the magnetic pellets in examples 3-4 to 3-6 using magnetic powder made of NdFeB alloy had the second greatest magnetism. As for the Fe₄(P₂O₇)₃ group (examples 3-7 to 3-9) and the reduced iron group (examples 3-1 to 3-3), the magnetism of the magnetic pellets was not so great, but the weak magnetism (<300 G) can let the magnetic pellets to be used in a digestive track, since they would be better dispersed to adherer onto the surface of the intestinal villi.

The saturated fatty acid having a melting temperature greater than 60° C. and being insoluble in water was used as the material of the first matrix. Therefore, the magnetic powder was isolated from the external environment, and hence did not contact the acid, base, oxidant and/or reductant in the external environment. As a result, the stability and workability of the magnetic pellet can be increased. Moreover, the saturated fatty acid can help to fix the position and alignment orientation of the magnetic powder. Accordingly, the magnetic dipole of the same direction can be formed after the magnetic pellets were magnetized to increase the magnetism of the magnetic pellets, and the application scope of the magnetic pellets can be enlarged.

### Embodiment 4

### Effect of Water Soluble Substance on the Appearance of the Magnetic Pellets

In this embodiment, a water soluble substance was added to the starting materials for preparing the porous magnetic pellets. First, ascorbic acid, magnetic powder, and MCC were uniformly mixed. 5 wt % of HPMC aqueous solution was then added and uniformly mixed again. Subsequently, extrusion and spheronization were performed to obtain wet magnetic pellets.

The wet magnetic pellets were shade dried for 72 hours to reduce 30%-50% of water content. Next, the drier magnetic pellets were immersed in 500 mL of 60% (v/v) EtOH aqueous solution for 6 hours to dissolve the ascorbic acid in the solution for producing more and larger pores. After draining, the wet magnetic pellets are dried at 50° C. for 12 hours. Next, the weight loss of the dried magnetic pellets was measured to evaluate the dissolved amount of ascorbic acid and the increased pore volume. The outward appearance was also observed.

TABLE 5
Effect of the water soluble substance (ascorbic acid)
on the outward appearance of the magnetic pellets

| | | | | | Dried | Weight | |
|---|---|---|---|---|---|---|---|
| | Pellet's | | | Ascorbic | pellet's | loss of | |
| | weight | Fe₄(P₂O₇)₃ | *matrix | acid | weight | pellets | Outward |
| Ex. | (mg) | (wt %) | (wt %) | (wt %) | (mg) | (wt %) | appearance |
| 4-1 | 200 | 10 | 10 | 80 | 125 | 37.5 | Dark brown |
| | | | | | | | material loss |
| | | | | | | | in trace |
| | | | | | | | amount |
| 4-2 | 200 | 10 | 20 | 70 | 143 | 28.5 | complete |
| 4-3 | 200 | 10 | 30 | 60 | 152 | 24.0 | complete |
| 4-4 | 200 | 10 | 40 | 50 | 158 | 21.0 | complete |
| 4-5 | 200 | 10 | 50 | 40 | 169 | 15.5 | complete |
| 4-6 | 200 | 10 | 60 | 30 | 173 | 13.5 | complete |

*MCC:HPMC = 95:5 (w/w).

In the process of preparing porous magnetic pellets, the magnetic pellets had been immersed in aqueous solution for a period of time, and the ascorbic acid was dissolved in the aqueous solution to produce additional pores or increase the original pore size. In Table 5, it can be clearly seen that the more the ascorbic acid was added, the more the weight loss of the magnetic pellets had. However, when the addition amount of the ascorbic acid was too much, such as 80 wt % in example 4-1, the outward appearance of the magnetic pellets could not maintain complete, and trace amount of dark brown material was lost.

### Embodiment 5

### Porous Magnetic Pellets

In this embodiment, porous magnetic pellets were prepared. The starting materials are listed in Table 6, and the preparations are described below.

Lactic acid (LA, slightly soluble in water), ascorbic acid, magnetic powder, and MCC were uniformly mixed. 5 wt % of HPMC aqueous solution was added and then uniformly mixed again. Subsequently, extrusion and spheronization were performed to obtain wet magnetic pellets. The wet magnetic pellets were shade dried for 72 hours to reduce water content. Then, the drier magnetic pellets were immersed in water for 24 hours to dissolve the ascorbic acid and then produce more and larger pores. Next, the magnetic pellets were dried at 180-300° C. for 4 hours to decrease the water content and form cyclic polylactate by polymerizing the lactic acid. Thus, the bondability and firmness of the magnetic pellets could be increased and the magnetic pellets were finally shaped.

TABLE 6
Starting materials of preparing magnetic pellets and measured
maximum magnetism after the magnetic pellets were magnetized.

| | | | | | | Ascorbic | Magne- |
|---|---|---|---|---|---|---|---|
| | Dia. | | HPMC | MCC | LA | acid | tism |
| Ex. | (mm) | SrO•6Fe₂O₃ | (g) | (g) | (g) | (g) | (G) |
| 5-1 | 2.0 | 500 | 50 | 450 | 0 | 0 | 135 |
| 5-2 | 2.0 | 500 | 50 | 100 | 350 | 0 | 118 |
| 5-3 | 2.0 | 500 | 50 | 0 | 350 | 100 | 110 |
| 5-4 | 2.0 | 500 | 50 | 100 | 0 | 350 | 122 |
| 5-5 | 1.2 | 500 | 50 | 450 | 0 | 0 | 129 |
| 5-6 | 1.2 | 500 | 50 | 100 | 350 | 0 | 122 |
| 5-7 | 1.2 | 500 | 50 | 0 | 350 | 100 | 102 |
| 5-8 | 1.2 | 500 | 50 | 100 | 0 | 350 | 113 |
| 5-9 | 1.0 | 500 | 50 | 450 | 0 | 0 | 131 |
| 5-10 | 1.0 | 500 | 50 | 100 | 350 | 0 | 110 |
| 5-11 | 1.0 | 500 | 50 | 0 | 350 | 100 | 101 |
| 5-12 | 1.0 | 500 | 50 | 100 | 0 | 350 | 108 |

From Table 6, it can be seen that the diameter of the magnetic pellets had a certain effect on the magnetism of the magnetic pellets. The larger the magnetic pellets, the greater the magnetism the magnetic pellets had. In addition, the more the addition amount of ascorbic was, the porosity the magnetic pellets had. However, the addition amount of the ascorbic acid had a little effect on the magnetism of the magnetic pellets.

### Embodiment 6

### Magnetic Pellets Having a Shell Layer

In this embodiment, the magnetic pellet core did not contain any magnetic powder, but the shell layer thereof contains magnetic powder. The starting materials and structure features are listed in Table 7.

TABLE 7
Starting materials and structure features of the magnetic pellets
having a shell layer

| | Magnetic pellet's | Shell's | Shell's weight | Magnetism* |
|---|---|---|---|---|
| Ex. | core structure | material | increase | (G) |
| 6-1 | Porous | HPMC | 30% | 0 |
| 6-2 | Porous | MP + | 10% | 55 |
| | | HPMC** | | |
| 6-3 | Porous | MP + | 30% | 87 |
| | | HPMC** | | |
| 6-4 | Solid | HPMC | 30% | 0 |
| 6-5 | Solid | MP + | 10% | 23 |
| | | HPMC** | | |
| 6-6 | Solid | MP + | 30% | 67 |
| | | HPMC** | | |

*Measured after the magnetic pellets were magnetized.
**Magnetic powder (MP):HPMC = 3:1 (w/w).

From Table 7, it can be known that the more the weight increase of the shell layer was, the greater the measured magnetism of the magnetic pellets was, as comparing with the control groups (examples 6-1 v. 6-4). However, the magnetism of the magnetic powder in the shell layers was weaker than the magnetic powder in the magnetic pellet's cores.

### Embodiment 7

### Magnetic Composite Pellets

In this embodiment, the magnetism of a single magnetic pellet (FIG. 1) and a magnetic composite pellet (FIG. 3), were compared. The results are listed in Table 8. From Table 8, it can be known that the magnetism of the magnetic pellet was greater than the magnetism of the magnetic composite pellet having the same diameters (examples 7-1 v. 7-3). However, no matter a single porous magnetic pellet or a magnetic composite pellet, the magnetism was increased as the diameter thereof was increased.

TABLE 8
Magnetism of the porous magnetic pellet and the magnetic
composite pellet

| | | | | Magnetism (G) | |
|---|---|---|---|---|---|
| | Magnetic | diameter | SrO•6Fe₂O₃ | Non- | |
| Ex. | Pellet's Type | (mm) | (wt %) | magnetized | Magnetized |
| 7-1 | Porous | 1.2 | 10-20 | 128 | 459 |
| 7-2 | magnetic | 1.0 | 10-20 | 103 | 387 |
| | pellet | | | | |
| 7-3 | Magnetic | 1.2 | 10-20 | 82 | 213 |
| 7-4 | composite | 2.0 | 17.5-22.5 | 105 | 289 |
| | pellet* | | | | |

*The magnetic composite pellet includes 40 wt % of solid magnetic pellets having a diameter of 0.3 mm.

In light of forgoing, since porous magnetic pellets could be prepared, the porous magnetic pellets can further adsorb active substance in a liquid state or in a suspension state, or the porous magnetic pellets can enter a digestive tract to adsorb the toxic substances in the digestive tract by their pores. Especially the pore size distribution of the porous magnetic pellets was quite wide, about 0.1-20 μm. Therefore, the porous magnetic pellets having various pore sizes can be used to adsorb various molecules with various sizes. In addition, the shell layer could be used to increase the stability of the porous magnetic pellets, and add different release functions to the porous magnetic pellets. Moreover, the diameter of the magnetic pellets can be varied according to the needs to have a wider application range.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A medical magnetic pellet, comprising:
a first matrix having pores and containing a modified cellulose, a starch or a combination thereof; and
magnetic powder distributed in the first matrix and made from a material which is magnetic or is induced to be magnetic.

2. The medical magnetic pellet of claim 1, wherein a content of the first matrix is 2.5-50 parts by weight.

3. The medical magnetic pellet of claim 2, wherein a content of the magnetic powder is 5-25 parts by weight.

4. The medical magnetic pellet of claim 1, wherein the pores have a diameter of 0.1-20 μm.

5. The medical magnetic pellet of claim 1, wherein the modified cellulose is hydroxypropyl methyl cellulose (HPMC), hydroxypropyl cellulose (HPC), hydroxyethyl cellulose (HEC), microcrystalline cellulose (MCC), or any combinations thereof.

6. The medical magnetic pellet of claim 1, wherein a material of the magnetic powder is an element, an oxide, an alloy, a salt, or any combinations thereof that contains at least one element of Fe, Co, Ni, Nd and Sr.

7. The medical magnetic pellet of claim 1, further comprising a first active substance distributed in the first matrix, the pores or a combination thereof, wherein the first active substance has at least a biological activity.

8. The medical magnetic pellet of claim 7, wherein a content of the first active substance is 30-70 parts by weight.

9. The medical magnetic pellet of claim 1, further comprising a shell layer coating on the first matrix.

10. The medical magnetic pellet of claim 9, wherein a material of the shell layer is a monosaccharide, a disaccharide, a sugar alcohol, an enteric polymer, or a fatty acid or an ester thereof having 5-50 carbons.

11. A medical magnetic composite pellet, comprising:
a second matrix; as well as
magnetic pellets distributed in the second matrix, wherein each of the magnetic pellets comprises:
a first matrix having pores and containing a modified cellulose, a starch, or a combination thereof; and
magnetic powder distributed in the first matrix and made from a material that is magnetic or is induced to be magnetic.

12. The medical magnetic composite pellet of claim 11, wherein the second matrix is made from water soluble small molecules, comprising a monosaccharide, an amino acid, an organic acid, a sugar alcohol, or any combinations thereof.

13. The medical magnetic composite pellet of claim 11, further comprising a second active substance distributed in the second matrix, wherein the second substance having at least a biological activity.

14. The medical magnetic composite pellet of claim 11, wherein the pores have a diameter of 0.1-20 μm.

15. The medical magnetic composite pellet of claim 11, wherein the modified cellulose is hydroxypropyl methyl cellulose (HPMC), hydroxypropyl cellulose (HPC), hydroxyethyl cellulose (HEC), microcrystalline cellulose (MCC), or any combinations thereof.

16. The medical magnetic composite pellet of claim 11, wherein a material of the magnetic powder is an element, an oxide, an alloy, a salt, or any combinations thereof that contains at least one element of Fe, Co, Ni, Nd and Sr.

17. The medical magnetic composite pellet of claim 11, further comprising a first active substance distributed in the first matrix, the pores or a combination thereof, wherein the first active substance has at least a biological activity.

18. The medical magnetic composite pellet of claim 11, further comprising a shell layer coating on the first matrix.

19. The medical magnetic composite pellet of claim 18, wherein a material of the shell layer is a monosaccharide, a disaccharide, a sugar alcohol, an enteric polymer, or a fatty acid or an ester thereof having 5-50 carbons.
